(19) United States
(12) Patent Application Publication
(10) Pub. No.: US 2017/0072151 A1
(43) Pub. Date: Mar. 16, 2017

(54) Rebreathing Devices

(71) Applicant: Albert DONNAY, Hyattsville, MD (US)

(72) Inventor: Albert DONNAY, Hyattsville, MD (US)

(21) Appl. No.: 15/125,285

(22) PCT Filed: Mar. 13, 2015
(86) PCT No.: PCT/US15/20443

§ 371 Date: Sep. 12, 2016

Related U.S. Application Data
(60) Provisional application No. 61/952,827, filed on Mar. 13, 2014.

(51) Int. Cl. A61M 16/00; A63B 23/18; A61M 16/06

(52) U.S. Cl. CPC A61M 16/0045; A61M 16/0688; A63B 23/18; A61M 2240/00

(57) ABSTRACT

A method of rebreathing for a human, comprising: breathing through a rebreathing device for up to 20 minutes at a time, repeated as needed; wherein said rebreathing device has a form of
a) cylindrical form made by rolling a sheet of paper, and able to fit snugly over either the mouth or the nose of a human in need of rebreathing;
b) cylindrical form made with a cardboard tube from a toilet paper roll;
c) cylindrical form made with a cardboard tube from a paper towel roll;
d) cylindrical form made from a washable, re-useable, solid tube and able to fit snugly over either the mouth or the nose;
e) rectilinear form made from a paper bag made of absorbent paper, at least 5 inches long [=12.7 cm] for adults, that is modified by i) cutting or tearing horizontally to shorten the bag so the remaining bag is approximately 5 to 7 inches [12.7 to 17.8, cm] tall, and ii) folding over one corner of the rectangular bottom approximately 1 inch [=2.5 cm] and cutting or tearing it off to make a hole approximately 1.5 inch [=3.8 cm] in diameter to allow rebreathing when worn over the nose and mouth;
f) triangular form folded in the shape of a mask able to cover both nose and mouth made from i) a sheet of paper size 8.5×11 inches [=21.6×28 cm], A-10, or any larger sheet of newspaper folded down to this size, or made by folding a foil-lined snack-food bag of approximately 6×10 inches [=15.2×25.4 cm]; or
g) conical form made from a paper, foam or plastic disposable cup, into which a hole is made in the bottom about 1 inch [=2.5 cm], which need not need to be centered, so that the original cup opening may be placed over the nose and mouth and pushed lightly against the face so that it forms a snug seal all around.

### CROSS REFERENCE TO RELATED APPLICATIONS

This application is the US national stage entry of PCT/US2015/020443, filed Mar. 13, 2015, which claims the benefit of U.S. Provisional Application No. 61/952,827, filed Mar. 13, 2014; and these applications are both incorporated herein by reference.

### BACKGROUND OF THE INVENTION

Field of the Invention

Rebreathing devices stimulate respiration, accelerate the exhalation of carbon monoxide, alcohol and other poisons, and provide a zero-impact reflexive method of aerobic exercise and weight loss.

Background

Carbon monoxide (CO) poisoning is commonly treated with normobaric and/or hyperbaric oxygen delivered at high flow rates via plastic non-rebreather masks and/or hoods designed to reduce carbon dioxide rebreathing as much as possible. But since these treatments are expensive; only available from physicians, emergency responders and hospitals; and increase the risk of delayed neurological sequelae up to five-fold by forcing more CO off hemoglobin into tissues, older methods were researched to find cheaper, more accessible, less toxic alternatives available without a prescription.

Dr. J S Haldane developed a portable suitcase-size device for reviving victims of CO poisoning in the 1920s that delivered a mixture of air and 3% to 5% carbon dioxide (CO₂) called “carbetha” from a tank via a non-rebreather mask. Research studies show breathing carbetha significantly increases minute ventilation (MV) and lowers venous carboxyhemoglobin as fast as does 100% oxygen. Since humans exhale 3% to 5% CO₂, partial-rebreather and CPAP masks were evaluated to determine their ability to achieve the same result. While tight-fitting plastic masks with a large dead space retain CO₂ and increase MV, they trap uncomfortable levels of heat and moisture, and their small ports make breathing difficult. Paper masks are more comfortable but lack dead space.

The author invented several rebreather device designs that optimize gas exchange at low cost. These include several cylindrical forms that can be used for rebreathing by either the mouth or the nose, and a variety of other forms that cover both nose and mouth simultaneously: a rectilinear form made from a shortened paper bag with one corner torn off; a conical form made from a disposable cup with a hole in the bottom, a triangular form made from folding paper or foil-lined snack food bags, and a historical form made with modern reconstructions of masks worn during the Middle Ages. All but the latter can be made to fit any size face from infant to adult.

Donnay's rebreathing devices are designed to revive respiration in moribund victims of carbon monoxide, opiate, alcohol, and other poisonings, speed up the elimination of these and other gaseous poisons from human bodies, reduce the severity of hangovers, and provide a zero-impact reflexive method of aerobic exercise and weight loss.

There are other types of rebreathing devices in past or present commercial use but they are designed for other purposes, function differently, and are clearly distinguished from the present invention by certain features. Among the other types are:
a) “full-rebreather” masks designed to fit snugly and scrub or remove CO₂ from a closed-loop system as it accumulates;
b) “partial-rebreather” masks designed to be loose-fitting with open ports that allow some exhaled CO₂ to escape while some is captured and rebreathed from a gas bag that is attached to the mask, but they do not control which portion of the exhalation is captured and are designed only for use with a supply of oxygen or other medical gas mixture;
c) “respiratory training” masks and hand-held devices designed to strengthen respiratory muscles through the use of resistance valves that increase the physical effort needed to breathe through them; and
d) the “respiratory stimulator,” U.S. Pat. No. 5,647,345, designed to increase minute ventilation for some of the same purposes as the present invention but it specifies a large 1.5 to 20 liter capacity, and it does not fit snugly to the face, allowing captured CO₂ to leak out.

In contrast to the present invention, in which the masks all have dead space volume equal or less than 0.5 liters, preferably 0.2, 0.3, 0.4 or 0.5 liters, the relatively larger dead space volume of these prior masks and the shape and location of their exhalation ports do not capture for rebreathing only the last portion of each exhalation, in which the CO₂ concentration is always highest. Other portions of exhaled breath have much lower CO₂ concentrations making them less effective at boosting minute ventilation. Only the present invention is designed to capture the highest possible concentration of CO₂ without adding any resistance that would make the work of breathing more difficult.

There also are other types of breathing devices designed to reduce CO levels in human bodies including:
a) the H-H Inhalator developed by Dr. Haldane in the 1920s that delivered a fixed concentration of CO₂ in air from a tank; and
b) the ClearMate that allows the user to set the concentration and flow rate of CO₂ mixed with oxygen.

But in contrast to the present invention, these are not rebreathing devices. Both incorporate a non-rebreather face mask and neither seeks to capture for rebreathing any of the CO₂ exhaled.

### SUMMARY OF THE INVENTION

An object of the invention is to provide a method of rebreathing for a human, comprising: breathing through a rebreathing device for up to 20 minutes at a time, optimally while lying down; however, the method also works while standing or sitting in any body position, including while running or bicycling or operating a motor vehicle.

The present invention provides a device and methods of breathing through such a device, including any of the embodiments of Donnay's rebreathing device described herein regardless of the exact methods and materials by which they are constructed, for up to 15 to 20 minutes at a time, repeated as needed and tolerated, in order to:

a) accelerate the elimination of carbon monoxide from tissues, blood and lungs after acute or chronic poisoning and thereby lower the total body burden of CO, whether CO is higher than normal due to increased endogenous CO production and/or reduced CO exhalation and/or from exposure to exogenous sources [such as from smoking or smoke inhalation, vehicle exhaust, or being around insufficiently vented combustion appliances burning wood, coal, biomass, gas or oil];

b) accelerate elimination of alcohol during or after drinking, and thereby more quickly lower one's blood alcohol level compared to not using the rebreathing device and, if done for at least 20 minutes just before sleeping, will also reduce the severity of any hangover upon waking:

c) accelerate elimination of any other ingested or inhaled toxin that is volatile in the blood, such as liquid pesticides or cleaning products; the fumes of mercury, formaldehyde, or lead; or drugs of abuse that have been deliberately injected, smoked, “huffed”; or “sniffed” such as acetone;

d) quickly increase minute ventilation and oxygen saturation in neonates, children and adults who are barely breathing and at risk of death from respiratory failure, even if unconscious, and whatever the cause [eg. alcohol or morphine poisoning, drowning, electrocution, shock. traumatic birth, etc], provided their lung tissues are still intact and their muscles of respiration still function:

e) stop attacks of anxiety and hyperventilation syndrome;

f) stop hiccups;

g) reduce the CO body burden in people with waxing and waning conditions that are associated with abnormally elevated levels of exhaled CO. These include multi-sensory sensitivity syndrome and other sensory disorders such as central sensitization syndrome, sensory processing disorder, multiple chemical sensitivity, tinnitus, blurred vision, loss of smell, and metallic taste; neurological disorders such as autism spectrum disorder, Asperger's syndrome. lupus, and multiple sclerosis; psychiatric conditions such as depression, post-traumatic stress disorder, schizophrenia, and manic depression [bi-polar]; movement and muscle disorders such as chronic fatigue syndrome [aka CFIDS or ME], fibromyalgia, irritable bowel disorder, parkinsonism and rheumatoid arthritis: respiratory disorders such as asthma and bronchitis; as well as premenstrual syndrome and the nausea and vomiting of pregnancy;

h) reduce the CO body burden in people experiencing side effects from medications that overlap with CO symptoms such as headache, fatigue, nausea, impotence, and photo-sensitivity; and

i) provide a zero-impact form of reflexive aerobic exercise that can be done lying down by athletes and musicians trying to increase their functional lung capacity, people trying to lose weight, and people who are bed-ridden, paralyzed, frail, recovering from illness or surgery, or suffering from movement disorders like Parkinson's for whom exercising their limbs is impossible or ill-advised.

### BRIEF DESCRIPTION OF THE FIGURES

FIG. 1 A is a plan view of the cylindrical form of the device made from a sheet of paper showing the first step in construction

FIG. 1 B is a perspective view of the cylindrical form of the device made from a sheet of paper showing the second step in construction

FIG. 1 C is a perspective view of the cylindrical form of the device made from a sheet of paper showing the third step in construction

FIG. 1 D is a perspective view of the cylindrical form of the device made from a sheet of paper showing the final construction

FIG. 1 E is a perspective view of the cylindrical form of the device made from a cardboard tube taken from a toilet paper roll after modification for use over the nose

FIG. 2 A is a plan view of the triangular form of the device made from a sheet of paper showing the first step in construction

FIG. 2 B is a plan view of the triangular form of the device made from a sheet of paper showing the second step in construction

FIG. 2 C is a plan view of the triangular form of the device made from a sheet of paper showing the third step in construction

FIG. 2 D is a plan view of the triangular form of the device made from a sheet of paper showing the fourth step in construction

FIG. 3 A is a plan view of the triangular form of the device made from a foil-lined bag showing the first step in construction

FIG. 3 B is a plan view of the triangular form of the device made from a foil-lined bag showing the second step in construction

FIG. 3 C is a plan view of the triangular form of the device made from a foil-lined bag showing the third step in construction

FIG. 3 D is a plan view of the triangular form of the device made from a foil-lined bag showing the fourth step in construction

FIG. 3 E is a plan view of the triangular form of the device made from a foil-lined bag showing the fifth step in construction

FIG. 3 F is a perspective view of the triangular form of the device made from a foil-lined bag showing the final construction

FIG. 4 shows a person lying down holding the triangular form of the invention in place for use as a rebreathing device.

### DETAILED DESCRIPTION

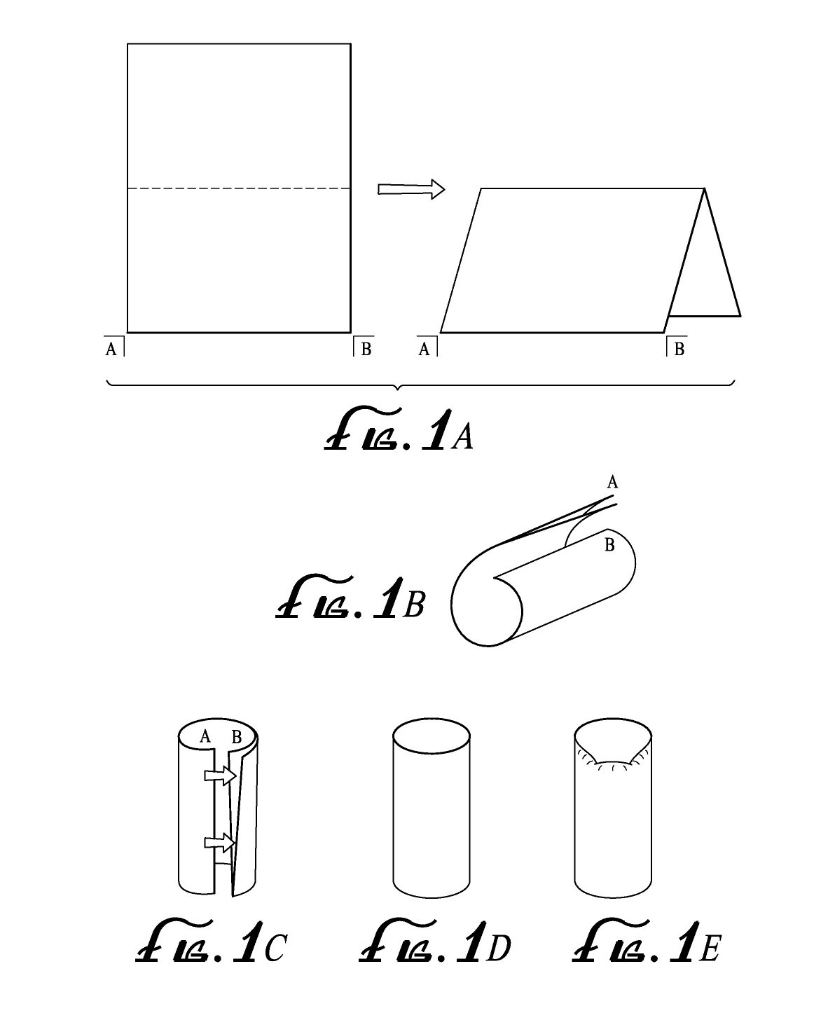

1. Cylindrical form made by rolling one sheet of approximately 8.5×11-inch [=21.6×28.0 cm] or A10 paper. To make the shortest size appropriate for children, hold the paper in portrait mode and fold it in half horizontally as shown in FIG. 1A. Then take the folded sheet [which can also be made by folding down newspapers or magazine pages] and roll up the longer edge loosely around two fingers to form a tube approximately 1 inch [=2.5 cm] in diameter as shown in FIG. 1B. In the last step, tuck the free end into the fold made by the other end as shown in FIG. 1C, which will secure the tube as shown in FIG. 1D so it does not unravel. This and the other cylindrical embodiments are all designed to be fit snugly over either the mouth or the nose. They all can be held in place there by hand, by tape, or by sonic strap around the head. To make longer devices appropriate for adults, fold over only one-third to one-fourth of the sheet from the end instead of folding it in half. Then roll it around three fingers instead of two to create a device that is closer to 1.5 inches [=3.8 cm] in diameter and tuck it the same way shown in FIGS. 1B, C and D. For babies, additional folds are needed to reduce the size of the sheet fold before rolling it, and then by rolling it only around one for babies. Preferably single-use-only for hygienic reasons.

2. Cylindrical form made with the cardboard tube taken from a toilet paper roll and used as is for adults, or cut shorter for infants and children. Fits better over mouth than nose. For nose use, one side of one end must be pushed in about ¼ to ½ inch [=0.6 to 1.3 cm] as shown in FIG. 1E. Single-use-only for hygienic reasons.

3. Cylindrical form made with the cardboard tube taken from inside of paper towel roll and cut to approximately 5 to 6 inch [=12.7 to 15.2 cm] length for adults, or shorter for children and much shorter for infants. Fits better over mouth than nose. For nose use, one side of one end must be pushed in about % to inch [=0.6 to 1.3 cm] as shown in FIG. 1E. Single-use-only for hygienic reasons.

4. Cylindrical form made from a washable, re-useable, solid tube made of any other material than cardboard with the same approximately 5.5×1.5-inch [=3.8×14.0 cm] dimensions and designed to fit snugly over either the mouth or the nose. Designed for multiple uses provided it is washed with soap and dried in-between.

5. Rectilinear form made from a paper lunch bag made of absorbent paper, at least 5 inches [=12.7 cm] long for adults, optimally made of Kraft-style paper and optimally size number 4 [=3×5×10.7 inches or 7.6×12.7×21.2 cm], that is modified by:
a) cutting or tearing it horizontally to shorten it so the remaining bag is approximately 5 to 7 inches tall [=12.7 to 17.8, cm], and
b) folding over one corner of the rectangular bottom approximately 1 inch [=2.5 cm] and cutting or tearing it off to make a hole approximately 1.5 inch [=3.8 cm] in diameter to allow rebreathing when worn over the nose and mouth. For maximum utility, the edge of the bag ending at the hole should be aligned over the nose. This embodiment can be taped or held snugly to the face but the paper material is not strong enough to support a head strap. Single-use-only for hygienic reasons.

6. Triangular form folded in the shape of a mask that covers both nose and mouth. One embodiment of this form can be made from a sheet of paper size 8.5×11 inches 22×28 cm], A-10, or any larger sheet of newspaper folded down to this size. The other embodiment, also described in this section, is made from folding a foil-lined snack-food bag of approximately 6×10 inches [=15.2×25.4 cm] in size.

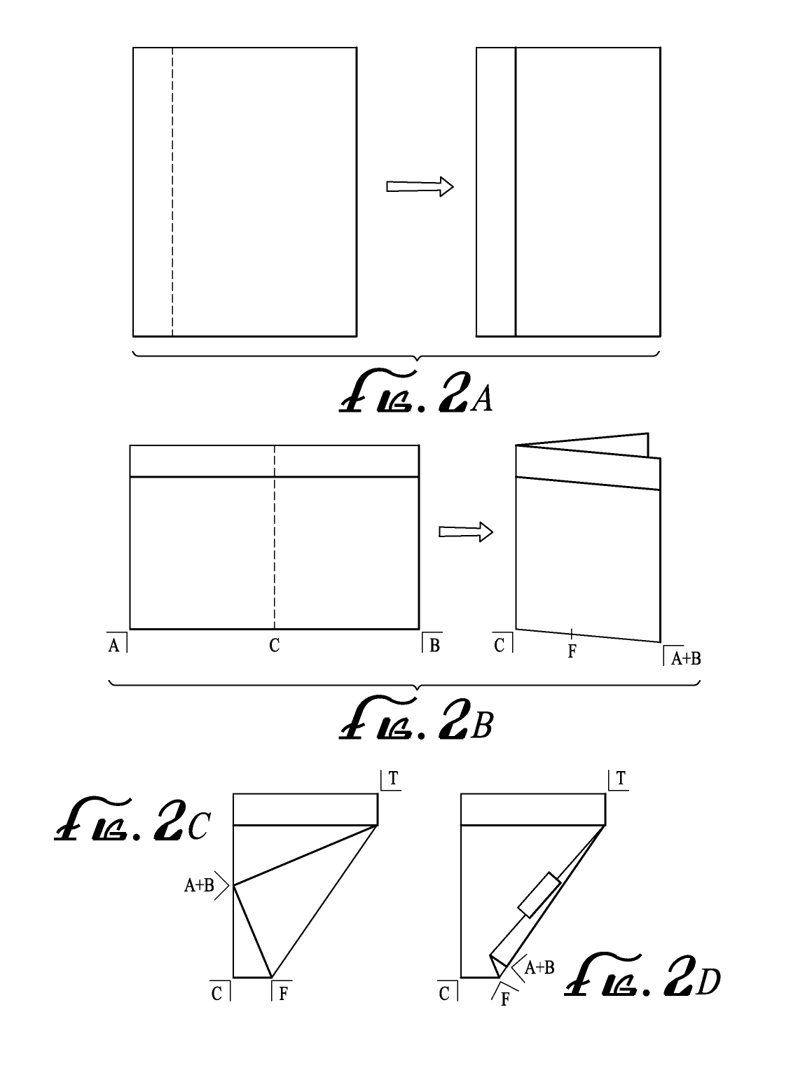

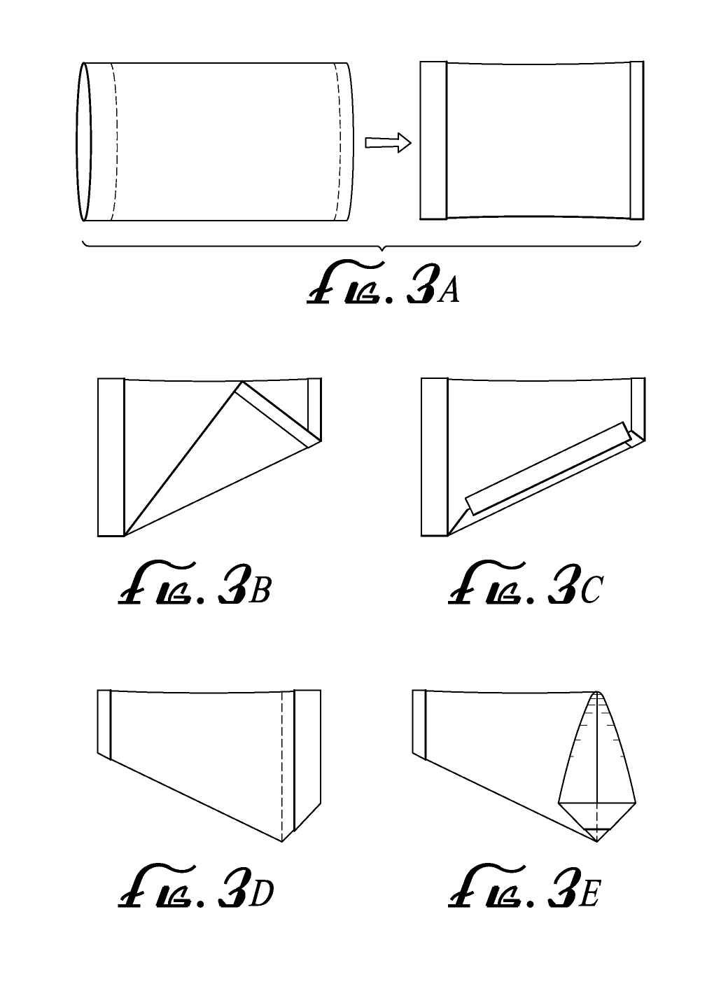

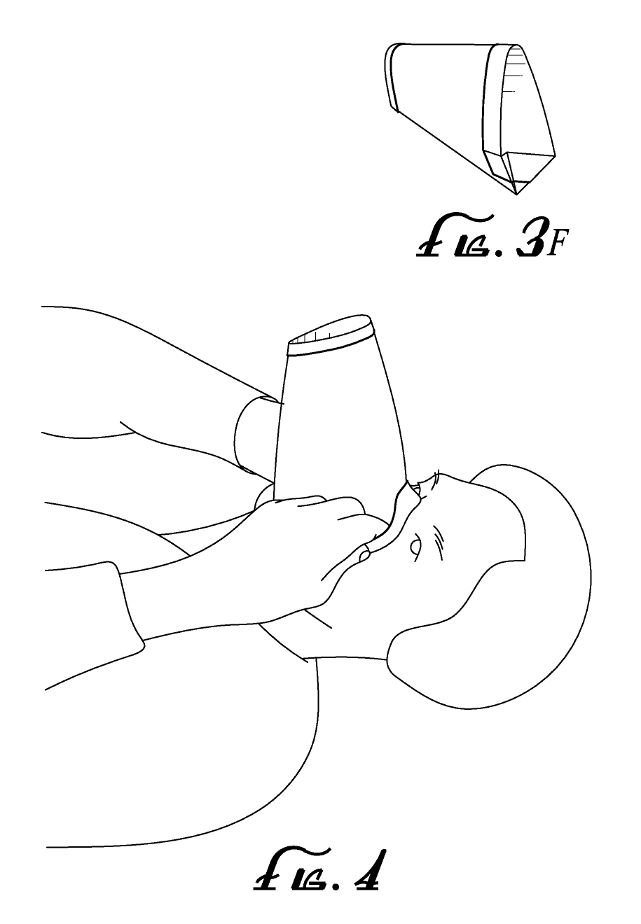

For the embodiment made from paper, the instructions are as follows:
a) Fold the long side of the sheet about 1.5 inches [=3.8 cm] in from the edge so it creates a collar of double thickness all the way across, as shown in FIG. 2A.
b) Turn the sheet over so this folded collar strip is once again visible along the top edge, as shown in FIG. 2B.
c) With the sheet in landscape orientation, fold the sheet in half vertically, so that the left edge lines up with right and the folded edge is again visible across the top.
d) Fold the lower right corner over until it just touches approximately the middle of the left edge and adjust this fold until it intersects the right edge just below the top band and the bottom edge about 2 inches [=5.1 cm] from the left corner, as shown in FIG. 2C.
e) Fold the right angle of this triangular flap back over to the right so that the longer edge lines up with the hypotenuse and then repeat this fold in the same fashion to form a tight and narrow seam. Crease this edge firmly and then secure it with a piece of tape as shown in FIG. 2D. It can also be secured with paper clips or staples.
f) To use the device, put one hand inside the larger end and spread the device open like a funnel so that it looks similar to the embodiment shown in FIG. 3F. Then open the smaller hole in the distal end. For optimal efficiency, users should lie on their back, grasp the device with their fingertips inside the collar formed by the folded over edge, place it over their nose and mouth, and then hold it snugly against their face by pushing gently on their cheeks with their fingertips as shown in FIG. 4. Like the cylindrical forms, it also can taped to the face or held in place with a head strap that is affixed to the collar on each side of the nose. Single-use-only for hygienic reasons.

For the embodiment made from a foil-lined snack-food bag approximately 6×10 inches [=15.2×25.4 cm], the instructions are:
g) Open both ends of the bag across their entire width without tearing the material as shown on the left side of FIG. 3A.
h) After emptying, cleaning and drying the inside of the bag, turn each end back on itself to form cuffs as shown on the right side of FIG. 3A. The larger cuff is approximately 1 to 1.5 [=2.5 to 3.8 cm] inches wide and the smaller is approximately half the larger.
i) With the bag lying flat as shown in FIG. 3A, fold the lower corner of the smaller cuff towards the opposite long side of the bag to form a flap in the shape of an approximately 30-60-90 degree triangle as shown in FIG. 3B. This fold should stop short of the wider cuff at the left end but should cross entirely through the narrower cuff on the right, leaving approximately 2 inches [=5.1 cm] open along this end. When this fold is correctly aligned, crease it firmly and then tightly roll up the triangular flap from the right angle corner towards the folded edge but not beyond it.
j) Put a piece of tape over the rolled up triangle to keep it from unrolling as shown in FIG. 3C. If no tape is available, the edge can be secured with a few staples or paper clips.
k) Turn the bag over and make a temporary light crease along the dotted line shown in FIG. 3D. Fold the lower corner of the wider cuff up and into the opening at the end of the bag so that it forms two small 45-45-90 degree triangles that are hidden and facing each inside the opening when the bag is smoothed flat, as shown in FIG. 3D). The further that this corner is folded up into the larger cuff, the narrow and smaller the resulting device becomes.
l) Fold the larger end of the bag so that it stands up 90 degrees along the temporary fold shown in FIG. 3D. Open this end of the bag and smooth down the 2 triangles formed at the bottom of the cuff as shown in FIG. 3E.
m) Put one hand inside the larger end and spread the device open like a funnel as shown in FIG. 3F. Then open the smaller hole in the distal end.
n) To use the device with maximum efficiency, the person lies down with their head facing up. They grasp the device with their fingertips inserted all the way into the larger cuff. Then they pull the device over their nose and mouth, and with their fingertips, push the cuff snugly their face as shown in FIG. 4. As with the triangular form made from a sheet of paper, this embodiment also may be taped to the cheeks or held in place with a head strap affixed to the cuff on each side of the nose. Preferably single-use-only for hygienic reasons unless thoroughly washed with soap and dried between uses.

7. Conical form made from paper, foam or plastic disposable cup into which a hole is made in the bottom about the size of a bottle cap. The bottom hole can be cut, poked, punched, or torn and does not need to be centered. After making the hole, the larger opening is placed over the nose and mouth and pushed lightly against the face so it forms a snug seal all around, similar to that shown in FIG. 4 for the triangular form. The cup can also be taped against the face or tied around the head. For adults, the appropriate size cup is 8 to 12 ounces [=236.6 to 354.9 ml]; for school children the cup should be approximately half this size, and for infants approximately one quarter the adult size. Preferably single-use-only for hygienic reasons unless thoroughly washed with soap and dried between uses.

8. Historical form made from reproductions of masks with extremely long noses that were worn by doctors in the Middle Ages to ward off Plague. These are available in leather, fabrics, plastic, paper, or paper mache. The long nose was originally designed to be stuffed with a mixture of herb and oils thought to prevent Plague, which increased resistance and made breathing through them difficult. Reproductions are sold without any stuffing and can be made into rebreather devices with a single modification.

The rebreather device of this invention may also be designed for single use, mass manufactured, packaged in multiples, and dispensed from a container.

The specific embodiments described above are exemplary and not intended to be limiting.

1. A method of rebreathing for a human, comprising: breathing through a rebreathing device for up to 20 minutes at a time, repeated as needed;
wherein said rebreathing device has a form selected from the group consisting of
a) cylindrical form made by rolling a sheet of paper, and able to fit snugly over either the mouth or the nose of a human in need of rebreathing;
b) cylindrical form made with a cardboard tube from a toilet paper roll;
c) cylindrical form made with a cardboard tube from a paper towel roll;
d) cylindrical form made from a washable, re-useable, solid tube and able to fit snugly over either the mouth or the nose;
e) rectilinear form made from a paper bag made of absorbent paper, at least 5 inches long [=12.7 cm] for adults, that is modified by i) cutting or tearing horizontally to shorten the bag so the remaining bag is approximately 5 to 7 inches [=12.7 to 17.8, cm] tall, and ii) folding over one corner of the rectangular bottom approximately 1 inch [=2.5 cm] and cutting or tearing it off to make a hole approximately 1.5 inch [=3.8 cm] in diameter to allow rebreathing when worn over the nose and mouth;
f) triangular form folded in the shape of a mask able to cover both nose and mouth made from i) a sheet of paper size 8.5×11 inches [=21.6×28 cm], A-10, or any larger sheet of newspaper folded down to this size, or made by folding a foil-lined snack-food bag of approximately 6×10 inches [=15.2×25.4 cm]; and
g) conical form made from a paper, foam or plastic disposable cup, into which a hole is made in the bottom about 1 inch [=2.5 cm], which need not need to be centered, so that the original cup opening may be placed over the nose and mouth and pushed lightly against the face so that it forms a snug seal all around.

2. The method of claim 1, wherein said triangular from is made from paper, as follows:
a) Fold the long side of the sheet about 1.5 inches [=3.8 cm] in from the edge so it creates a collar of double thickness all the way across, as shown in FIG. 2A.
b) Turn the sheet over so this folded collar strip is once again visible along the top edge, as shown in FIG. 2B.
c) With the sheet in landscape orientation, fold the sheet in half vertically, so that the left edge lines up with right and the folded edge is again visible across the top.
d) Fold the lower right corner over until it just touches approximately the middle of the left edge and adjust this fold until intersects the right edge just below the top band and the bottom edge about 2 inches [=5.1 cm] from the left corner, as shown in FIG. 2C.
e) Fold the right angle of this triangular flap back over to the right so that the longer edge lines up with the hypotenuse and then repeat this fold in the same fashion to form a tight and narrow seam. Crease this edge firmly and the secure it with a piece of tape as shown in FIG. 2D. It can also be secured with paper clips or staples.
f) To use the device, put one hand inside the larger end and spread the device open like a funnel so that it looks similar to the embodiment shown in FIG. 3F. Then open the smaller hole in the distal end. For optimal efficiency, users should lie on their back, grasp the device with their fingertips inside the collar formed by the folded over edge, place it over their nose and mouth, and then hold it snugly against their face by pushing the gently on their cheeks with their fingertips as shown in FIG. 4. Like the cylindrical forms, it also can taped to the face or held in place with a head strap that is affixed to the collar on each side of the nose. Single-use-only for hygienic reasons.

3. The method of claim 1, wherein triangular form f) is made from a foil-lined snack-food bag approximately 6×10 inches [=15.2×25.4 cm], as follows:
g) Open both ends of the bag across their entire width without tearing the material as shown on the left side of FIG. 3A.
h) After emptying, cleaning and drying the inside of the bag, turn each end back on itself to form cuffs as shown on the right side of FIG. 3A. The larger cuff is approximately 1 to 1.5 inches [=2.5 to 3.8 cm] wide and the smaller is approximately half the larger.
i) With the bag lying flat as shown in FIG. 3A, fold the lower corner of the smaller cuff towards the opposite long side of the bag to form a flap in the shape of an approximately 30-60-90 degree triangle as shown in FIG. 3B. This fold should stop short of the wider cuff at the left end but should cross entirely through the narrower cuff on the right, leaving approximately 2 inches [=5.1 cm] open along this end. When this fold is correctly aligned, crease it firmly and then tightly roll up the triangular flap from the right angle corner towards the folded edge but not beyond it.
j) Put a piece of tape over the rolled up triangle to keep it from unrolling as shown in FIG. 3C. If no tape is available, the edge can be secured with a few staples or paper clips.
k) Turn the bag over and make a temporary light crease along the dotted line shown in FIG. 3D. Fold the lower corner of the wider cuff up and into the opening at the end of the bag so that it forms two small 45-45-90 triangles that are hidden and facing each inside the opening when the bag is smoothed flat, as shown in FIG. 3D. The further that this corner is folded up into the larger cuff, the narrow and smaller the resulting device becomes.
l) Fold the larger end of the bag so that it stands up 90 degrees along the temporary fold shown in FIG. 3D. Open this end of the bag and smooth down the 2 triangles formed at the bottom of the cuff as shown in FIG. 3E.
m) Put one hand inside the larger end and spread the device open like a funnel as shown in FIG. 3F. Then open the smaller hole in the distal end.
n) To use the device with maximum efficiency, the person lies down with their head facing up. They grasp the device with their fingertips inserted all the way into the larger cuff. Then they pull the device over their nose and mouth, and with their fingertips, push the cuff snugly their face as shown in FIG. 4. As with the triangular form made from a sheet of paper, this embodiment also may be taped to the cheeks or held in place with a head strap affixed to the cuff on each side of the nose. Preferably single-use-only for hygienic reasons unless thoroughly washed with soap and dried between uses.

4. The method of claim 1, wherein the rebreather is sized for a child.

5. The method of claim 1, wherein the rebreather is held in place by a head strap when in use.

6. The method of claim 1, wherein the rebreather is held in place by tape when in use.

7. The method of claim 4, wherein the rebreather is sized for an infant.

8. The method of claim 1, wherein the rebreather device is incorporated into a hat, helmet, hood or other head covering so that it can be used without having to hold or tape it in the proper position.

9. The method of claim 1, wherein the rebreather device is incorporated into a face mask or face shield so that it can be used without having to hold or tape it in the proper position.

10. The method of claim 1, wherein the rebreather device is designed for single use, mass manufactured, packaged in multiples, and dispensed from a container.

11. The method of claim 1, wherein the rebreather device is used while lying down.

12. The method of claim 1, wherein the rebreather device is used while running.

13. The method of claim 1, wherein the rebreather device is while riding a bicycle.

14. The method of claim 1, wherein the rebreather device is used while sitting.
